(19) United States
(12) Patent Application Publication
(10) Pub. No.: US 2016/0310287 A1
(43) Pub. Date: Oct. 27, 2016

(54) Artificial Disc and Method for Relief of Lower Back Pain

(71) Applicant: Donald Steven Corenman, Edwards, CO (US)

(72) Inventor: Donald Steven Corenman, Edwards, CO (US)

(21) Appl. No.: 15/139,908

(22) Filed: Apr. 27, 2016

Related U.S. Application Data
(60) Provisional application No. 62/153,166, filed on Apr. 27, 2015.

(51) Int. Cl. A61F 2/44

(52) U.S. Cl. CPC A61F 2/442; A61F 2210/0076

(57) ABSTRACT

The invention provides artificial disc devices for treating lower back pain. The invention also provides a method for implanting said artificial disc devices that is minimally invasive and less dangerous.

### CROSS-REFERENCE TO RELATED APPLICATIONS

This application claims priority to U.S. Provisional Patent Application No. 62/153,166 filed Apr. 27, 2015, the contents of which are incorporated by reference herein in its entirety.

### BACKGROUND OF THE INVENTION

The normal discs of the lumbar spine are designed to absorb shock, but allow movement that places the spine in positions to help with the activities of daily living. Unfortunately, the lumbar spine is often subjected to degenerative disc disease, resulting in the breakdown of the ability of the lumbar disc to absorb shock. This degeneration increases stress to the surrounding spine, especially in the bones of the vertebral body.

When this degeneration occurs, the increased stress can cause significant lower back pain. The common method of surgically fusing the degenerative segment works well for pain relief but restricts motion, making the procedure less desirable.

Lumbar artificial disc replacement has been used for some years to reduce stress to the vertebral segment, while allowing some motion. However, a common problem with such replacements is that they must be implanted from the front of the spine, which necessitates an anterior approach to implant the artificial disc.

The anterior approach is fraught with potential complications, including scarring of the great vessels (aorta and vena cava), potential damage to the small and large intestines, the potential of retrograde ejaculation in males, damage to the ureters, and abdominal wall muscle injury. In addition, when these artificial discs fail, the approach to remove or replace them also requires the same anterior approach. In some instances, removal or replacement of these artificial discs can be life threatening.

Thus, there is an urgent need for an improved intervertebral disc replacement and method of implantation for safer and more effective treatment of lower back pain. The present invention addresses these needs.

### SUMMARY OF THE INVENTION

In one aspect, the present invention relates to an artificial disc device, comprising: a core having a height, a width, a depth, a top surface, and a bottom surface; a first endplate comprising at least one tine affixed to the top surface of the core; and a second endplate comprising at least one tine affixed to the bottom surface of the core; wherein the first and the second endplates are substantially parallel to each other, and wherein the at least one tine of the first and second endplates protrudes in a direction away from the core.

In one embodiment, the at least one tine is attached to a deployment mechanism. In one embodiment, the deployment mechanism is a rotatable rod. In one embodiment, the deployment mechanism can be actuated to switch the at least one tine between a retracted configuration and a protruding configuration. In one embodiment, the device further comprises a locking mechanism for securing one artificial disc device to another artificial disc device. In one embodiment, the locking mechanism is selected from the group consisting of a latch, a setting adhesive, or a magnet.

In one embodiment, the core comprises a material selected from the group consisting of an elastic synthetic medical polymer, rubber, silicone rubber, polyvinyl alcohol, polyurethane resin, polysiloxane modified styrene-ethylene/butylene block copolymer, and ultra high molecular weight polyethylene. In one embodiment, the first and the second endplates comprise a material selected from the group consisting of metal, plastic, polymer, stainless steel, aluminum, titanium, aluminum alloy, titanium alloy, polyetheretherketone, ultra high molecular weight polyethylene, and metal alloy coated with ultra high molecular weight polyethylene.

In another aspect, the present invention relates to a threaded artificial disc device, comprising: a core having a height, a width, a depth, a top surface, and a bottom surface; a first endplate affixed to the top surface of the core, wherein the first endplate is at least partially threaded; and a second endplate affixed to the bottom surface of the core, wherein the second endplate is at least partially threaded; wherein the first and the second endplates are substantially parallel to each other, and wherein the threading of the first and the second endplates are aligned.

In one embodiment, a surface of the core not in contact with the first or second endplate is at least partially threaded. In one embodiment, the device comprises a cylindrical shape. In one embodiment, the core comprises a material selected from the group consisting of an elastic synthetic medical polymer, rubber, silicone rubber, polyvinyl alcohol, polyurethane resin, polysiloxane modified styrene-ethylene/butylene block copolymer, and ultra high molecular weight polyethylene. In one embodiment, the first and the second endplates comprise a material selected from the group consisting of metal, plastic, polymer, stainless steel, aluminum, titanium, aluminum alloy, titanium alloy, polyetheretherketone, ultra high molecular weight polyethylene, and metal alloy coated with ultra high molecular weight polyethylene.

In another aspect, the present invention relates to a minimally invasive method for implanting an artificial disc device in a subject to treat lower back pain, the method comprising the steps of: making an incision in the annulus fibrosis of the intervertebral disc; removing a volume of nucleus pulposus substantially equal to the volume of an artificial disc device of the present invention; and inserting the artificial disc device into the space previously occupied by the volume of removed nucleus pulposus. In one embodiment, the incision is made laterally to the intervertebral disc. In one embodiment, the incision is made posteriorly to the intervertebral disc.

In another aspect, the present invention relates to a minimally invasive method for implanting a threaded artificial disc device in a subject to treat lower back pain, the method comprising the steps of: making an incision in the annulus fibrosis of the intervertebral disc; drilling a cavity into the nucleus pulposus with a drill bit having substantially the same diameter and threading as a threaded artificial disc device of the present invention; and screwing in the threaded artificial disc device into the cavity; wherein the threaded artificial disc device is oriented such that each endplate is separately in contact with the upper and lower vertebral bone. In one embodiment, the incision is made laterally to the intervertebral disk. In one embodiment, the incision is made posteriorly to the intervertebral disc.

In another aspect, the present invention relates to a kit for use in the treatment of lower back pain, the kit comprising at least one artificial disc device comprising a core having a height, a width, a depth, a top surface, and a bottom surface; a first endplate comprising at least one tine affixed to the top surface of the core; and a second endplate comprising at least one tine affixed to the bottom surface of the core; wherein the first and the second endplates are substantially parallel to each other, and wherein the at least one tine of the first and second endplates protrudes in a direction away from the core.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A and FIG. 1B depict two exemplary embodiments of an artificial disc device.

FIG. 2A through FIG. 2C depict the tines of an artificial disc device and their mechanism of use.

FIG. 3A through FIG. 3C depict exemplary modes of implanting exemplary artificial disk devices.

FIG. 4A through FIG. 4C depict an exemplary embodiment of threaded artificial disc devices and exemplary modes of implanting the same.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical artificial disc devices and methods of use. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

The present invention includes an artificial disc device for the relief of lower back pain. The present invention also includes improved methods of implanting intervertebral disc devices, including the artificial disc device of the present invention.

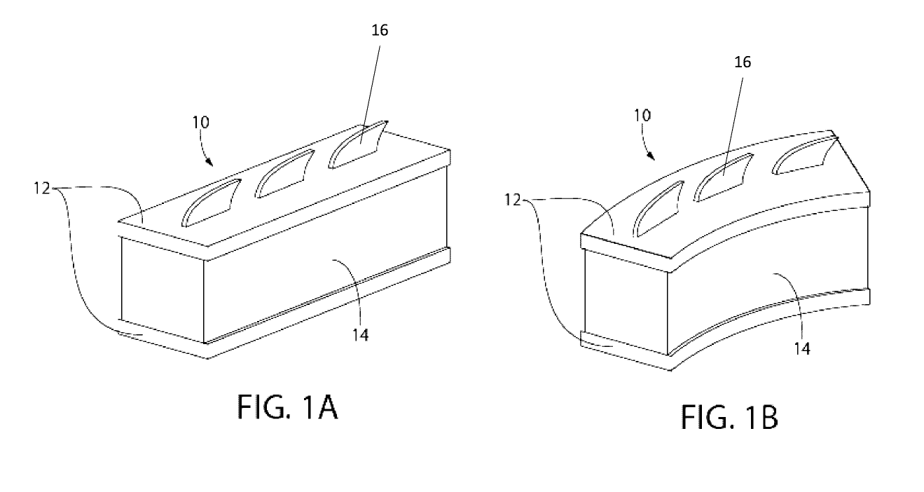

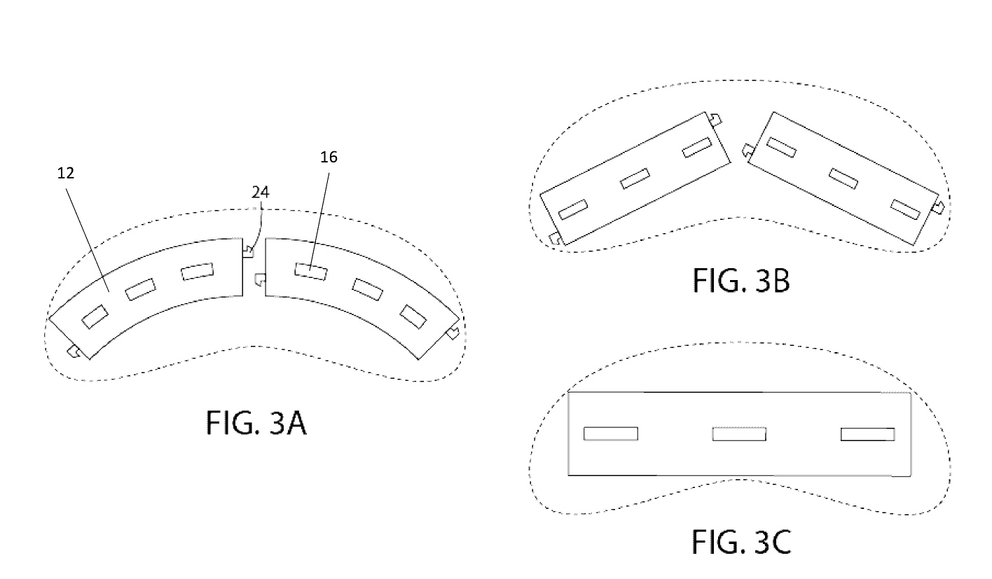

Referring now to FIG. 1A, an exemplary artificial disc device 10 is shown. Artificial disc device 10 comprises two endplates 12 and a core region 14. As shown, core region 14 is generally sandwiched between two substantially parallel endplates 12. Device 10 may take any shape desired, such as a substantially straight configuration (FIG. 1A), or a substantially curved or curvilinear configuration (FIG. 1B). In certain embodiments, the ends of artificial disc device 10 may comprise a push-to-lock mechanism 24 (FIG. 3A, FIG. 3B). Push-to-lock mechanism 24 may be any mechanism that is able to secure one component to another upon pushing the two components against each other, such as a touch latch, a magnetic mechanism, a setting adhesive, or any other mechanism that secures two components upon physical contact, as would be understood by those skilled in the art. It should be appreciated that device 10 is not limited to any particular shape or size, and can be constructed in any shape or size to fit the needs of a subject requiring the replacement device and the medical professional performing the procedure.

Core 14 has a height, width, and depth that is sized preferably smaller than a recipient's intervertebral disc to enable the use of an artificial disc device 10 in minimally invasive implant surgery. For example, in certain embodiments, the overall artificial disc device may have a height, measured from the bottom endplate to the top endplate, of 1 mm, 2 mm, 3 mm, 4 mm, 5 mm, 6 mm, 7 mm, 8 mm, 9 mm, or 10 mm. In certain embodiments, the artificial disc device may have a width of 1 mm, 2 mm, 3 mm, 4 mm, 5 mm, 6 mm, 7 mm, 8 mm, 9 mm, or 10 mm. In certain embodiments, the artificial disc device may have a width of up to 20 mm.

Core 14 is constructed from a biocompatible material that is shock absorbing and allows three-dimensional movement, such that it mimics or provides a functionality that is substantially equivalent to the properties of a normal intervertebral disc in terms of compressibility, elasticity, torsion, and the like. The material of core 14 may include, but is not limited to any elastic medical-grade synthetic polymer, rubber, silicone rubber, polyvinyl alcohol, polyurethane resin, and the like. The material of core 14 may also include, but is not limited to any appropriate biocompatible thermoplastic material, such as Flex® (Concept, Inc.), Pellethane® (DOW Chemical), polysiloxane modified styrene-ethylene/butylene (SEBS) block copolymer, ultra high molecular weight polyethylene (UHMWPE), polyetheretherketone (PEEK), and the like, without limitation, provided such materials are selected and configured to replicate equivalent mechanical properties in naturally occurring intervertebral discs, as would be understood by those skilled in the art.

Endplates 12 may be constructed from any suitable material, such as any biocompatible or non-bioreactive materials, including but not limited to metals, plastics and other polymers commonly used to construct spinal implants. For example, such materials may include, without limitation, stainless steel, aluminum, titanium, aluminum or titanium alloys, PEEK, UHMWPE, or metals or metal alloys coated with a material such as UHMWPE. In other embodiments, endplates 12 may be manufactured to include a bioactive material, such as an alkali-containing ceramic (glass, glass-ceramic, or crystalline) material that reacts as it comes in contact with physiological tissues to stimulate bone formation. It should be appreciated that there is no limitation to the material construction of device 10, as contemplated herein.

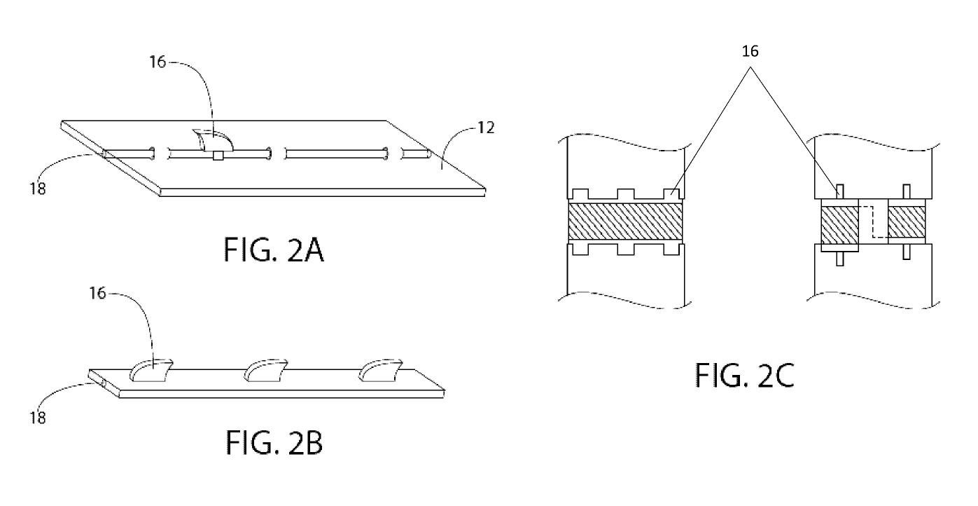

In various embodiments, endplates 12 comprises tines 16 to stabilize artificial disc device 10 after implant (FIG. 1A, FIG. 1B). Tines 16 may be fixed or deployable. In a deployable embodiment, tines 16 may be attached to a rod mechanism 18 running along at least a portion of the length of endplates 12, such that endplates 12 may have a retracted configuration (FIG. 2A) and a deployed configuration (FIG. 2B). When in a retracted configuration, tines 16 lay flush or recessed within the surface of endplates 12. Rod mechanism 18 may be actuated to rotate tines 16 into a deployed configuration. It should be appreciated that in some embodiments, core 14, endplate 12, or both may include one or more reciprocal recessed regions, grooves or pockets for tines 16 to retract into in order for tines 16 to create a smooth or non-protruding surface when in the retracted configuration. Tines 16 may be manufactured from any suitable material as described similarly for endplates 12.

Referring now to FIG. 3A through FIG. 3C, exemplary modes of implanting the artificial disc devices of the present invention are shown. Contrary to current methods of implanting artificial discs from an anterior direction, the implant devices described herein can be inserted via small incisions in the annulus fibrosis from a directly lateral direction (FIG. 3B) or from a posterior direction (FIG. 3A). The incision is just large enough to permit entry for an artificial disc device. After the incision is made, a volume of nucleus pulposus approximately equal to the volume of an artificial disc device is removed, and the artificial disc device is inserted in its place. In one embodiment, a plurality of artificial disc devices may be implanted into one intervertebral disc space. Device 10 may include one or more holes, recesses, grooves or pockets for engaging an insertion tool, such that the insertion tool can suitably engage, position and release device 10 through the small incision.

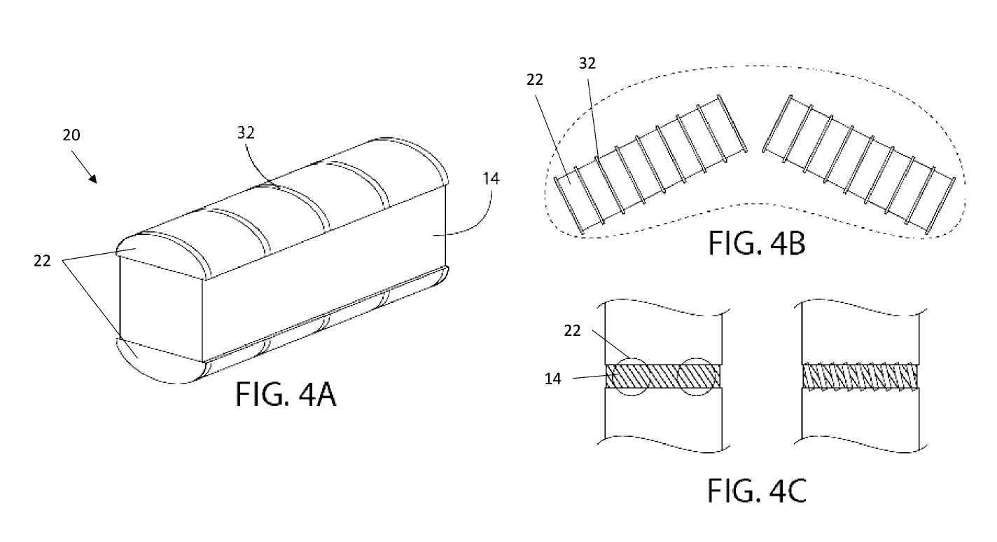

Referring now to FIG. 4A, an exemplary threaded artificial disc device 20 is depicted. Artificial disc device 20 comprises endplates 22 comprises screw thread 32 on at least a portion of its outer surface. In the embodiment depicted in FIG. 4A, screw threads 32 are not continuous. Preferably, screw threads 32 are dimensioned and aligned as if they were part of a single continuous thread to enable the device 20 to be screwed into a cylindrical cavity. In another embodiment (not pictured), core 14 is threaded such that a substantially continuous screw thread 32 wraps around device 20.

Referring again to the exemplary embodiments depicted in FIG. 4A and FIG. 4B, implantation can also be made via small incisions in the annulus fibrosis from a directly lateral direction or from a posterior direction (FIG. 4C). The incision is just large enough to permit entry for a threaded artificial disc device. After the incision is made, a drill bit comprising the same diameter and threading of the threaded artificial disc device is used to drill a cavity into the nucleus pulposus, and the threaded artificial disc device is screwed into place. Preferably, the threaded artificial disc device is oriented such that each endplate 32 is separately in contact with the upper and lower vertebral bone (FIG. 4C). In one embodiment, a plurality of threaded artificial disc devices may be implanted into one intervertebral disc space.

In any method of the present invention, the incisions made in the annulus fibrosis are intended to preserve as much of the natural structure of the annulus fibrosis as possible. Preferably, the angle of approach when making incisions from a directly lateral direction or from a posterior direction avoids the facet joints of the spine. Furthermore, implanting the devices of the present invention from a directly lateral direction or from a posterior direction avoids disturbing the great vessels, decreasing risk to the patient if it is necessary to remove the devices from an anterior direction.

The invention also includes a kit comprising one or more implant device and/or one or more components useful to perform the implantation procedure. For example, in addition to the kit including one or more replacement disc devices, the kit can further include one or more additional components, such as rongeurs, curettes, distractor instruments, and the like. In certain embodiments, the one or more instruments of the kit are sterile and contained in one or more individual sterile packages. The sterile kit described herein is thus immediately ready for surgery upon removal from the packages without pre-operation processing. In certain embodiments, the kit may include instructional material that describes, for instance, suggestions for determining the proper sized disc device and/or suggestions regarding the method of insertion. Instructional material may include a publication, a recording, a diagram, or any other medium of expression which can be used to communicate the usefulness of the device or implant kit described herein. The instructional material of the kit of the invention may, for example, be affixed to a package which contains one or more instruments which may be necessary for the desired procedure. Alternatively, the instructional material may be shipped separately from the package.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An artificial disc device, comprising:
a core having a height, a width, a depth, a top surface, and a bottom surface;
a first endplate comprising at least one tine affixed to the top surface of the core; and
a second endplate comprising at least one tine affixed to the bottom surface of the core;
wherein the first and the second endplates are substantially parallel to each other, and wherein the at least one tine of the first and second endplates protrudes in a direction away from the core.

2. The device of claim 1, wherein the at least one tine is attached to a deployment mechanism.

3. The device of claim 2, wherein the deployment mechanism is a rotatable rod.

4. The device of claim 2, wherein the deployment mechanism can be actuated to switch the at least one tine between a retracted configuration and a protruding configuration.

5. The device of claim 1, further comprising a locking mechanism for securing one artificial disc device to another artificial disc device.

6. The device of claim 5, wherein the locking mechanism is selected from the group consisting of a latch, a setting adhesive, or a magnet.

7. The device of claim 1, wherein the core comprises a material selected from the group consisting of an elastic synthetic medical polymer, rubber, silicone rubber, polyvinyl alcohol, polyurethane resin, polysiloxane modified styrene-ethylene/butylene block copolymer, and ultra high molecular weight polyethylene.

8. The device of claim 1, wherein the first and the second endplates comprise a material selected from the group consisting of metal, plastic, polymer, stainless steel, aluminum, titanium, aluminum alloy, titanium alloy, polyetheretherketone, ultra high molecular weight polyethylene, and metal alloy coated with ultra high molecular weight polyethylene.

9. A threaded artificial disc device, comprising:
a core having a height, a width, a depth, a top surface, and a bottom surface;
a first endplate affixed to the top surface of the core, wherein the first endplate is at least partially threaded; and
a second endplate affixed to the bottom surface of the core, wherein the second endplate is at least partially threaded;
wherein the first and the second endplates are substantially parallel to each other, and wherein the threading of the first and the second endplates are aligned.

10. The device of claim 9, wherein a surface of the core not in contact with the first or second endplate is at least partially threaded.

11. The device of claim 9, wherein the device comprises a cylindrical shape.

12. The device of claim 9, wherein the core comprises a material selected from the group consisting of an elastic synthetic medical polymer, rubber, silicone rubber, polyvinyl alcohol, polyurethane resin, polysiloxane modified styrene-ethylene/butylene block copolymer, and ultra high molecular weight polyethylene.

13. The device of claim 9, wherein the first and the second endplates comprise a material selected from the group consisting of metal, plastic, polymer, stainless steel, aluminum, titanium, aluminum alloy, titanium alloy, polyetheretherketone, ultra high molecular weight polyethylene, and metal alloy coated with ultra high molecular weight polyethylene.

14. A minimally invasive method for implanting an artificial disc device in a subject to treat lower back pain, the method comprising the steps of:
making an incision in the annulus fibrosis of the intervertebral disc;
removing a volume of nucleus pulposus substantially equal to the volume of an artificial disc device of claim 1; and
inserting the artificial disc device into the space previously occupied by the volume of removed nucleus pulposus.

15. The method of claim 14, wherein the incision is made laterally to the intervertebral disc.

16. The method of claim 14, wherein the incision is made posteriorly to the intervertebral disc.

17. A minimally invasive method for implanting a threaded artificial disc device in a subject to treat lower back pain, the method comprising the steps of:
making an incision in the annulus fibrosis of the intervertebral disc;
drilling a cavity into the nucleus pulposus with a drill bit having substantially the same diameter and threading as a threaded artificial disc device of claim 9; and
screwing in the threaded artificial disc device into the cavity;
wherein the threaded artificial disc device is oriented such that each endplate is separately in contact with the upper and lower vertebral bone.

18. The method of claim 17, wherein the incision is made laterally to the intervertebral disk.

19. The method of claim 17, wherein the incision is made posteriorly to the intervertebral disc.

20. A kit for use in the treatment of lower back pain, the kit comprising at least one artificial disc device comprising a core having a height, a width, a depth, a top surface, and a bottom surface; a first endplate comprising at least one tine affixed to the top surface of the core; and a second endplate comprising at least one tine affixed to the bottom surface of the core; wherein the first and the second endplates are substantially parallel to each other, and wherein the at least one tine of the first and second endplates protrudes in a direction away from the core.
